(19) United States
(12) Patent Application Publication
(10) Pub. No.: US 2005/0183168 A1
(43) Pub. Date: Aug. 18, 2005

(54) Plants having decreased dormancy period and methods producing the plants

(76) Inventors: Hiroaki Kisaka, Kawasaki-shi (JP); Tetsuya Miwa, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/059,687

(22) Filed: Feb. 17, 2005

(30) Foreign Application Priority Data
Feb. 17, 2004 (JP) 2004-039659

(51) Int. Cl. A01H 1/00; C12N 15/82

(52) U.S. Cl. 800/287; 435/468

(57) ABSTRACT

The present invention provides a plant having a decreased dormancy period, a plant having a decreased dormancy period and uniform budding and/or germination timing, and a method for producing these plants. The intracellular 2-OG content in the plant may be increased, for example, by overexpressing an intracellular GDH gene in the plant. More specifically, the intracellular 2-OG content in the plant may be increased due to the overexpression of the GDH gene, by introducing into the plant a nucleic acid construct capable of enhancing expression of the GDH gene, particularly a nucleic acid construct capable of expressing the GDH gene.

### BACKGROUND OF THE INVENTION

1. Field of the Invention

The present invention relates to plants having a decreased dormancy period, and more particularly, to plants having a decreased dormancy period and an increased yield.

In addition, the present invention also relates to methods for producing plants having a decreased dormancy period, and particularly to methods for producing plants having a decreased dormancy period and an increased yield.

2. Brief Description of the Related Art

Dormancy refers to a state wherein the living plant will not germinate even if environmental conditions such as moisture, temperature, etc. are favorable for budding or germination of seeds. Dormancy is common in seeds, bulbs, deciduous fruit trees, or the like.

Dormancy is considered to be an important function for protecting a plant from inferior environments and for maintaining the plant seed. However, in modern agriculture, culture environments have improved with the widespread use of greenhouse cultivation or multi-cultivation, and thus dormancy may greatly hinder cultivation. For this reason, species of many plants which have a short or nonexistant dormancy period have been actively cultivated, and excellent results have been obtained.

In addition, a method for breaking plant dormancy has been developed using artificial treatment. It is considered that dormancy is mainly caused by the accumulation of germination inhibitors such as abscisic acid. To awake from dormancy, it is considered that a decrease of germination inhibitor or an increase in gibberellin, which exhibits the germination inhibitor-antagonistic action, is necessary. Therefore, the effects of dormancy breaking are expected in gibberellin treatments to the plant.

In fact, gibberellin induces germination of dormant seeds of oats, lettuce, etc., and dormant buds of potato, peach, camellia, etc. (Tomokazu Koshiba and Yuji Kamiya, “Science of new plant hormone”, Kodansha Ltd., 2002). It has been acknowledged that it has a remarkable effect to improve the germination of seeds of rice, family of wheat, ramie, peanut, Bromus catharticus, Bromus inermis Leyss., Japanese radish, Brassica, lettuce, aubergine, beefsteak plant, gloxinia, kalanchoe, Primula, Nigella damascene, etc., and also strawberry, Aralia cordata Thunberg, etc. (Vegetable horticulture great dictionary, Yokendo, 1977). Though the correlation between gibberellin and germination is not clear, onion, scallion, spinach, flower bulb, etc. are also regarded as representative plants which can go dormant.

The potato is also a plant which goes dormant, and budding of the potato cannot be observed for about 3 months after harvesting. However, as described above, it was reported that the dormancy period can be shortened by a gibberellin treatment (Tokushima Test and Research Report of Agriculture, vol. 36, pp. 7-17, 2000) and the potato is considered to be an appropriate material for examining the correlation between dormancy regulators and endogenous gibberellin content.

The potato has been cultivated in about 20 million ha (hectare), and its average yield is 1.48 tons per 10 a (are). The total yield is about three hundred million tons. The largest planted area is in Russia, followed by China and Poland, and the total yields of these areas are also in this order. The Japanese planted area for potatoes is about 130 thousand ha and its yield is about 4 million tons. Japan's potato consumption is 44% for starch raw materials, 21% for raw food, and 13% for processed food. However, as the import liberalization for corn or wheat comes into effective, low cost imports for starch will become available. Therefore, to compete in this new environment, it will be necessary to ensure a production system which maintains high quality and high quantity simultaneously by further improving potato cultivation. Specifically, it will be necessary to increase the average starch value from 13% to 18% and increase the average production yield per 10 a from 4 to 7 tons, and to further reduce production cost to half by increasing.

The starch value at the initial stage of tuber thickening after tuber formation is approximately 0.8%, and thereafter, this value increases with additional tuber thickening. Accordingly, in order to increase the starch value of tubers, it is important to provide sufficient time for tuber thickening. In addition, since the starch value rapidly decreases as the soil temperature increases, a soil temperature (10 cm underground) of 17 to 22° C. is an optimum temperature range for the tuber thickening period. For this reason, it is desirable to adjust the cultivation time so that the temperature at the flowering time, when tuber formation initiates, is about 18° C. More specifically, it is recommended that spring planting is occurs 7 to 10 days before the air temperature reaches 10° C., and autumn planting occurs 10 days before the average temperature reaches 23° C.

Alternatively, as for the tuber yield, it is preferable to ensure a sufficient tuber thickening period, and that the yield of the potato increases by about 60 to 70 kg per day per 10 a during the thickening period. Furthermore, it is desirable to plant in the farm field as soon as possible in view of the optimum temperature for the starch value.

In addition, generally, the the budding period, which is the time from the tuber to the bud sprout, varies, and variations of 15 days are not unusual. In general, as compared with the yield of stumps which bud earlier, the yield of stumps which bud later decreases, since there is not enough time for tuber thickening. If the budding is delayed for 15 days, it is estimated that the yield per plant decreases up to 24%. In this way, the variation of the budding period is considered to be a significant factor in the decrease of the yield. Therefore, fast and uniform budding from tubers, that is, small growth variations are an essential factor for increasing the yield. (Potato encyclopedia, Minoru Yoshida, Rural Culture Association, 1988).

Gibberellin is snthesized through two pathways; one is a mevalonic acid pathway in which gibberellin is synthesized from acetic acid-derived mevalonic acid via isopentenyl diphosphate, and the other is a non-mevalonic acid pathway in which gibberellin is synthesized by isopentenyl diphosphate produced from glucose-derived pyruvic acid and glyceraldehyde-3-phosphate. Isopentenyl diphosphate is converted to tetracyclic hydrocarbon ent-kaurene via geranyl diphosphate, geranylgeranyl diphosphate, and ent-copalyl diphosphate. Then, ent-kaurene is subjected to three successive oxidations to generate ent-kaurenoic acid, which is subjected to three successive oxidations to synthesize GA12. The biosynthesis following the GA12 synthesis is catalyzed by dioxygenase utilizing 2-oxoglutarate as a co-substrate. At present, 100 or more varieties of free gibberellins are registered, but active gibberellins which have physiological activity are only a portion of them (GA1, GA3, GA4, etc.).

For the purpose of studying the physiological effects of gibberellin, it was attempted to introduce into a plant genes involved in gibberellin synthesis. Huang et al. produced a transformant by introducing the 20-oxidase gene isolated from Arabidopsis into Arabidopsis in the sense direction, where the gene was overexpressed. It has been reported that this transformant shows increased content of GA1, GA9, and GA20, advanced flowering time, and a decreased dormancy period of the seeds, compared with the nontransform ants. However, it has also been reported that there is a remarkable morphological abnormality in a plant where the extension of hypocotyls and stems were observed (Plant Physiology, 118: 773-781, 1998). Moreover, Carrera et al. isolated from potatoes a gene encoding GA20-oxidase (StGA20ox1), and introduced it into potatoes in the sense direction and in the antisense direction. It was reported that, when it was introduced in the sense direction and overexpressed, the length of an internode increased and its budding time from tubers was advanced (Plant Journal, 22: 247-256, 2000). However, in a transformant in which a gene encoding GA20-oxidase was introduced in the antisense direction, the dormancy period was reduced, and the number and weight of tubers were greatly reduced, and thus enhancement of the yield was not obtained. Furthermore, since no difference was observed between the budding time from potato tubers in which a gene encoding GA20-oxidase was introduced in the antisense direction and the budding time from tubers of the nontransformant, the authors suggested that there was no correlation between GA20-oxidase and dormancy.

Alternatively, independent of the above-described study, a transgenic plant was produced having a glutamate dehydrogenase (GDH) gene introduced, and an E. coli-derived NADP-dependent GDH gene (gdhA) was introduced into tobacco and corn for the purpose of giving phosphonoglycine herbicide resistance. Based on these studies, it was reported that its dry weight, total amino acid content, and water-soluble carbohydrate content were significantly increased (Lightfoot et al., Canada, CA2180786, 1996). Similarly, Tian et al. (China, CN00109779. 2) reported that tobacco exhibited good growth results by introducing a Neurospora-derived NADP-dependent GDH gene thereinto, when compared with tobacco into which the gene had not been introduced. Furthermore, it was reported that an Aspergillus nidulans-derived NADP-dependent GDH gene was introduced into tomatoes, thereby increasing a free amino acid content in the fruit (Japanese Unexamined Patent Publication No. 2001-238556, Kisaka et al.) and the same gene was introduced into potatoes, thereby increasing the weight and tuber number (JP2000404322). However, none of these reports describe a change in gibberellin content, nor an investigation of dormancy.

### SUMMARY OF THE INVENTION

The inventors of the present invention previously reported that the intracellular 2-oxoglutaric acid (hereinafter, “2-OG”) content is increased by overexpression of a glutamate dehydrogenase (hereinafter, “GDH”) gene in plant cells (Kisaka et al., Japanese Patent Application No. 2003-198559). GDH catalyzes the reversible reaction of incorporating ammonia into 2-OG to generate glutamic acid, and inversely releasing ammonia from glutamic acid to generate 2-OG However, in general, it is considered that GDH decomposes glutamic acid into 2-OG and ammonia, rather than incorporating ammonia into 2-OG for generating glutamic acid in the cells because GDH has a high Km value for ammonia, and as a result, the 2-OG content is increased.

On the other hand, the present inventors have considered that the dormancy period may be decreased by increasing the endogenous gibberellin activity in a plant, which results in advanced germination timing, thereby obtaining a strain having growth uniformity, and thus the yield can be increased. More particularly, the present inventors have found that since the biosynthesis following GA12 synthesis is catalyzed by dioxygenase which utilizes 2-oxoglutaric acid (2-OG) as a co-substrate, 2-OG is stably oversupplied by overexpressing a glutamate dehydrogenase (GDH) gene of the plant and the gibberellin activity in tubers can be enhanced, and that, as a result, strains can be actually obtained which have a decreased dormancy period, advanced budding or germination timing and uniform budding or germination timing, and therefore, strains having growth uniformity can be obatined. The present inventors have also found that the yield were increased.

It is an object of the present invention to provide a method for producing a plant comprising increasing the intracellular 2-OG content in said plant, and selecting a plant which has a decreased dormancy period as compared with the dormancy period of a naturally occurring plant of the same species.

It is further object of the present invention to provide the method as described above, wherein said plant has uniform budding or germination timing.

It is an object of the present invention to provide a method for producing a plant comprising overexpressing an intracellular GDH gene in said plant, and selecting a plant which has a decreased dormancy period as compared with the dormancy period of a naturally occurring plant of the same species.

It is a further object of the present invention to provide the method as described above, wherein said plant has uniform budding or germination timing.

It is a further object of the present invention to provide the method as described above, wherein a nucleic acid construct capable of enhancing the expression of the GDH gene is introduced into said plant.

It is a further object of the present invention to provide the method as described above, wherein a nucleic acid construct capable of expressing the GDH gene is introduced into said plant.

It is a further object of the present invention to provide the method as described above, wherein said nucleic acid construct comprises a nucleic acid molecule encoding the polypeptide comprising the amino acid sequence selected from the group consisting of SEQ ID NO: 2 and SEQ ID NO: 4.

It is a further object of the present invention to provide the method as described above, wherein said nucleic acid construct hybridizes under stringent conditions to a nucleic acid molecule selected from the group consisting of SEQ ID NO: 1 and SEQ ID NO: 3, and wherein said nucleic acid molecule encodes a polypeptide having GDH activity.

It is a further object of the present invention to provide the method as described above, wherein said plant is selected from the group consisting of oat, lettuce, potato, peach, camellia, rice, wheat, ramie, peanut, Bromus catharticus, Bromus inermis Leyss., Japanese radish, Brassica, aubergine, beefsteak plant, gloxinia, kalanchoe, Primula, Nigella damascena, strawberry, Aralia cordata Thunberg, onion, scallion, spinach, and flower bulb.

It is a further object of the present invention to provide the method as described above, wherein said plant is a potato.

It is also an object of the present invention to provide a plant which has an increased intracellular 2-OG content and a decreased dormancy period as compared with the intracellular 2-OG content and dormancy period of a naturally occurring plant of the same species.

It is a further object of the present invention to provide the plant as described above, wherein said plant has uniform budding or germination timing.

It is a further object of the present invention to provide a plant which has a decreased dormancy period as compared with the dormancy period of a naturally occurring plant of the same species, wherein said plant overexpresses an intracellular GDH gene.

It is a further object of the present invention to provide the plant as described above, wherein said plant has uniform budding or germination timing.

It is a further object of the present invention to provide the plant as described above, wherein said GDH gene comprises a nucleotide sequence encoding the polypeptide comprising the amino acid sequence selected from the group consisting of SEQ ID NO: 2 and SEQ ID NO: 4.

It is a further object of the present invention to provide the plant as described above, wherein said GDH gene comprises a sequence capable of hybridizing under stringent conditions to the nucleic acid molecule selected from the group consisting of SEQ ID NO: 1 or SEQ ID NO: 3, and wherein said gene encodes a polypeptide having GDH activity.

It is a further object of the present invention to provide the plant as described above, wherein said plant is selected from the group consisting of oat, lettuce, potato, peach, camellia, rice, wheat, ramie, peanut, Bromus catharticus, Bromus inermis Leyss., Japanese radish, Brassica, aubergine, beefsteak plant, gloxinia, kalanchoe, Primula, Nigella damascena, strawberry, Aralia cordata Thunberg, onion, scallion, spinach, and flower bulb.

It is a further object of the present invention to provide the plant as described above, wherein said plant is a potato.

Plant dormancy not only restricts the cultivation time but also is an obstacle to determining the growth and yield of the plant. Therefore, according to the present invention, by decreasing the plant dormancy period without causing significant disorders, restrictions to cultivation time can be removed and it allows the advancement of budding or germination timing, which results in uniform budding or germination. Therefore, the present invention can provide uniform growth of the plant and, consequently can provide simple cultivation and high yield, growth, and quality.

### BRIEF DESCRIPTION OF THE DRAWINGS

FIG. 1 is a schematic illustration of the constructed plasmid vector.
Nos-Pro : Nopaline synthase promoter
NPTII: Neomycin phosphotransferase
Nos-Ter: Nopaline synthase terminator
35S-Pro: Cauliflower mosaic virus 35S promoter
Mtd-AN-GDH: Aspergillus nidulans-derived glutamate dehydrogenase (GDH) having a transit peptide for mitochondria
HPT: Hygromycin phosphotransferase
H: HindIII, B: BamfI, X: XbaI, Sp: SpeI, E: EcoRI,
LB: Left Border, RB: Right Border

FIG. 2 shows the results of PCR for a transgenic potato using An-GDH gene specific primer (A), NPTII gene specific primer (B). Lane 1: 100 bp marker, Lane 2: nontransgenic potato, Lane 3: transgenic potato Mtd 1, Lane 4: transgenic potato Mtd 2, Lane 5: transgenic potato Mtd 3, Lane 6: transgenic potato Mtd 5, and Lane 7: transgenic potato Mtd 8.

FIG. 3 shows the results of Northern blotting for a transgenic potato. A total amount of 10 μg RNA extracted from tubers was subjected to electrophoresis and stained with ethidium bromide. The full length of an An-GDH gene is used for probe. Lane 1: nontransgenic potato, Lane 2: transgenic potato Mtd 1, Lane 3: transgenic potato Mtd 2, Lane 4: transgenic potato Mtd 3, Lane 5: transgenic potato Mtd 5, and Lane 6: transgenic potato Mtd 8.

FIG. 4 shows the 2-oxoglutarate (2-OG) content of GDH transgenic potato (n=3). Control: nontransgenic potato, and Mtd8: transgenic potato.

FIG. 5 shows the assay results for gibberellin from GDH transgenic potato tuber. Cont: sample solution from nontransgenic potato tuber, Mtd8: sample solution from nontransgenic potato tuber, Mtd8+Dami: sample solution from transgenic potato tuber+Daminozide solution. After 0-M: tuber immediately after harvesting, After 2-M: tuber 2 months after harvesting, and After 3-M: tuber 3 months from harvesting.

FIG. 6 shows the results of budding test for GDH transgenic potato tuber. (A) light treatment: for 3 days at 24° C. in a greenhouse and then transplanted on vermiculite; (B) Non-treatment: transplanting on vermiculite without light treatment.

### DESCRIPTION OF THE PREFERRED EMBODIMENTS

The present invention relates to a plant and its progenies which have a decreased dormancy period as compared with the dormancy period of naturally occurring plants of the same species, whereby a GDH gene in the plant cells is overexpressed, and a method for producing the plant.

Furthermore, the present invention provides a plant and its progenies capable of overexpressing a glutamate dehydrogenase (GDH) gene in the plant cells, and which have a decreased dormancy period as compared with the dormancy period of naturally occurring plants of the same species. The present invention also provides a plant and its progenies which have uniform budding and/or germination timing, and also a method for producing a plant having a decreased dormancy period as compared with that of naturally occurring plants of the same species, and which have uniform budding and/or germination timing, whereby a GDH gene is overexpressed in the plant cells.

In the present specification, the “naturally occurring plant of the same species” refers to the plant which belongs to the same species as that being produced by the present invention, and is observed in nature, and which has not been artificially manupilated or generated as a result of artificial manupilation.

More specifically, in one embodiment according to the present invention, a plant may be obtained that can overexpress a GDH gene and has a decreased dormancy period as compared with that of the naturally occurring plants of the same species, by introducing a gene capable of enhancing the expression of the GDH gene into the plant.

In one embodiment according to the present invention, by introducing a nucleic acid construct including GDH into a plant, GDH is expressed in the transformed plant to increase the 2-OG content, and the gibberellin activity is also increased. As a result, a plant may be obtained which has a decreased dormancy period and uniform budding or germination timing. GDH catalyzes the reaction of oxidatively deaminating glutamic acid to generate ammonia as well as the reverse reaction of generating glutamic acid by condensation of 2-OG and ammonia. However, it is generally considered that GDH catalyzes the decomposition of glutamic acid, that is, the reaction producing of 2-OG and ammonia in vivo preferentially, because the Km value for ammonia is high. In general, an excess amount of intracellular gibberellin, or the rapid increase in the level of intracellular gibberellin, may have a harmful influence on the growth or yield. However, according to the present invention, gibberellin is not provided extracellularly, rather the endogenous gibberellin level is increased via the increase of the 2-OG level due to the overexpression of the GDH gene. Therefore, according to the present invention, the activity of endogenous gibberellin would increase slowly through the increase of 2-OG, which results in a decrease of the dormancy period and the synchronization of budding or germination timing without causing significant harmful effects such as morphological abnormalities. Typically, crops are harvested simultaneously for all of the plants cultivated under substantially the same conditions for a predetermined period, which leads to the delay and diversity of budding or germination timing, which may directly cause a low yield in harvest. Therefore, according to the present invention, typically, additional stable improvement of the yield can be achieved.

According to the present invention, to increase the 2-OG content in the plant and, as a result, to raise the gibberellin activity, the method describes not only the introduction of the GDH gene, but also a gene encoding an enzyme, which may differ from GDH, but may be used to generate 2-OG from a glutamic acid. In a plant into which such a gene has been introduced, the 2-OG content will increase, and as a result, the gibberellin activity will be raised. For example, those enzymes may include aspartate aminotransferase, alanine aminotransferase, or the like.

Although the source of enzymes which can increase 2-OG content, e.g., GDH, used in the present invention is not particularly limited, an enzyme from Aspergillus genus, e.g., GDH from Aspergillus genus, may be more preferable. More preferably, the enzyme is the GDH from Aspergillus nidulans or Aspergillus awamorii. Additionally, a modified enzyme or its gene having a deletion of 1 or more amino acids, substitution, and insertion, e.g., modified GDH and modified GDH gene may be used in the present invention, as long as the modified enzyme has the enzyme activity as described above, e.g., GDH activity.

To overexpress one or more of these genes, expression of the genes endogenous to the plants may be enhanced instead of direct introduction of a nucleic acid construct which can express these genes. For example, such procedures include the introduction of a cis-acting nucleic acid construct which increases the endogenous gene expression, including introduction of a powerful promoter and/or enhancer, and the introduction of a trans-acting factor which can enhance the expression of these genes.

Furthermore, the localization of GDH in an intracellular organelle of the plant cell is not limited to the cytoplasm, but GDH may be localized in mitochondria, chlorophyl, or peroxysome, etc. Therefore, GDH having a signal or transit peptide attached to the N-terminal or C-terminal for transporting the enzyme protein in these intracellular organelles can also be used in the present invention.

These genes or cDNA can be easily produced by those skilled in the art, according to the published sequences. For example, the cDNA nucleotide sequence of GDH gene of Aspergillus nidulans is published in GenBank as Accession No. X16121. The nucleotide sequence (cDNA sequence) of Aspergillus nidulans derived GDH gene is shown in SEQ ID NO: 1 and the amino acid sequence of GDH in SEQ ID NO: 2. On the basis of these sequences, the GDH cDNA may be easily obtained by synthesizing a PCR primer which may amplify a DNA fragment including the part encoding the protein sequence and performing RT-PCR using the RNA isolated from Aspergillus nidulans culture as a template. Aspergillus nidulans can be obtained, including those registered as ATCC10074 or ATCC1 1267 or the like in American Type Culture Collection. In addition, the cDNA sequence of Aspergillus awamorii GDH cDNA is also published in GenBank as Accession No. Y15784. Aspergillus awamorii is registered in American Type Culture Collection as ATCC10548 or ATCC11358 or the like, and GDH cDNA can be obtained by the method described above using the obtained strains. The nucleotide sequence (cDNA sequence) of the GDH gene derived from Aspergillus awamorii is designated to SEQ ID NO: 3 and the GDH amino acid sequence is shown in SEQ ID NO: 4.

In the present invention, a nucleic acid construct which contains nucleic acid molecules which encode a polypeptide of SEQ ID NO: 2 or 4 may be used. Moreover, it is also possible to use a nucleic acid construct, including nucleic acid molecules, which have homology of at least 70% or more, preferably 80% or more, more preferably 90% or more, to SEQ ID NO: 1 or 3, wherein the nucleic acid construct encoding polypeptide shows GDH activity. This homology can be calculated using a program well-known to those skilled in the art, e.g., FASTA, with standard parameters. For example, programs such as FASTA etc. are provided by the Center for Information Biology and DNA Data Bank of Japan, National Institute of Genetics (DDBJ/CIB) (http://www.ddbj.nig.ac.jp/Welcome-j.html) (e.g., it can be used with score: 100, and alignment: 100). Other algorithms and programs for calculating homology among 2 sequences or more using standard parameters are well known to those skilled in the art together. These nucleic acid molecules can also include those capable of hybridizing to these sequences under “stringent conditions”, particularly to the sequences shown in SEQ ID NO: 1 or 3. The “stringent conditions” as used herein, refer to conditions under which so-called specific hybrids are formed and nonspecific hybrids are not formed. It is difficult to clearly express this condition numerically, but for example, such conditions can be defined as that under which DNA molecules having homology of 70% or more are preferentially hybridized, and other DNAs having homology of 70% or less are not significantly hybridized. More specifically, it may be defined as the condition under which hybridization may occur under normal washing conditions of southern hybridization of 50° C., 2×SSC and 0.1% SDS, preferably, 1×SSC, and 0.1% SDS, and more preferably, 0.1×SSC and 0.1% SDS. Moreover, any equivalent conditions may be apparent by those skilled in the art (e.g., Sambrook et al., Molecular Cloning: A Laboratory Manual, 2nd Edition, Cold Spring Harbor Laboratory Press, Cold Spring Harbor, N.Y., 1989; Ausubel et al., Current Protocols in Molecular Biology, John Wiley & Sons, Inc., 1994). Although a gene which is capable of hybridizing under these conditions may contain stop codon(s) within the gene or may have a mutation in its activity center which can cause the loss of activity, such a gene can easily be removed by determining the enzyme activity after connecting it to a commercially available expression vector.

SEQ ID NO: 1 and SEQ ID NO: 3 have a homology of 66%, as calculated by Genetyx Ver3.2. Similarly, SEQ ID NO: 2 and 4 were shown to have a homology of 87%.

For example, the GDH activity may be measured by the method of Ameziane et al. (Plant and Soil, 221: 47-57, 2000). More specifically, plant tissues, e.g., leaf, are cryopreserved with liquid nitrogen, ground using a mortar, and mixed with an extraction buffer {200 mM of Tris (pH 8.0), 14 mM of β-mercaptoethanol, 10 mM of L-Cysteine HCl, and 0.5 mM of PMSF} of 5×-volume of the tissue sample by weight. The resulting mixture is transferred to a centrifugal tube, centrifuged for 30 minutes at 12,000 rpm at 4° C., and the supernatant is ultrafiltered (Millipore, ultrafree 0.5 filter unit, Biomax-10), and the residue is washed several times with the extraction buffer. The resulting extracted enzyme solution is added to a solution containing 100 mM of Tris (PH 8.5), 20 mM of 2-oxoglutaric acid, 10 mM of CaCl₂, 0.2 mM of NADH, and 200 mM of NH₄Cl. Then, the GDH activity may be determined by measuring the decrease in the absorbance at 340 nm. The protein concentration in the extracted crude enzyme solution can be measured by a well-known method such as the Bradford method using, for example, bovine serum albumin (BSA) as a standard.

Furthermore, in the present invention, the method of raising the gibberellin activity by increasing 2-OG content is not limited to the introduction of GDH gene. A plant having high gibberellin activity due to the increase of 2-OG content can be also produced by introducing an enzyme which produces 2-OG from glutamic acid and which may differ from GDH. Examples of these enzymes include aspartate aminotransferase, alanine aminotransferase, or the like.

The nucleic acid construct used in the present invention can be prepared by any method well-known to those skilled in the art. Molecular biological methods, including isolation and determination of a nucleic acid construct sequence, may be referred to in publications, such as Sambrook et al., Molecular Cloning-Laboratory Manual, the 2nd edition, Cold Spring Harbor Laboratory Press). Alternatively, a gene amplification method such as PCR may be required for producing the nucleic acid construct which may be used in the present invention. These methods also may be referred to in publications such as F. M. Ausubel et al.(Current Protocols in Molecular Biology, John Wiley & Sons, Inc.(1994)).

The nucleic acid construct encoding GDH or the enzymes as described above may generally contain an appropriate promoter for plant cells, for example, a promoter for nopaline synthase gene, 35S promoter of cauliflower mosaic virus (CaMV35S), or appropriate terminators such as a terminator for nopaline synthase gene, and others as required, or a beneficial sequence for expression such as an enhancer and a marker gene for screening transformants such as a drug resistance gene including Kanamycin resistance gene, G418 resistance gene, and hygromycin resistance gene.

A promoter used in such a nucleic acid construct may be a constitutive promoter, tissue specific promoter, or growth stage specific promoter, and may be selected based on the the chosen promoter, according to the host, the required expression level, the expression target organ, or growth stage. In a preferred embodiment of the present invention, strong promoters which are non-specific to tissue and growth stage may be used, including, for example, the CaMV35S promoter. Tissue specific promoters include, for example, the phaseolin gene promoter, patatin gene promoter, or the like. In the most preferred embodiment of the present invention, a nucleic acid construct is used, wherein the GDH gene is driven by a powerful constitutive promoter such as the CaMV35S promoter.

The gene transfer method of the present invention is not particularly limited, and an appropriate gene transfer method for plant cells or plants known to those skilled in the art, depending the host, can be used. For example, in one embodiment of the present invention, a gene transfer method using Agrobacteruim is used. In such a transformation system, it is preferable to use a binary vector. In the case of Agrobacterium, the nucleic acid construct used in transformation further includes a T-DNA region which flanks the DNA sequence to be introduced. In a preferred embodiment of the present invention, the introduced sequence is inserted between the left and right T-DNA border sequences. Appropriate design and construction of such a transformation vector based on T-DNA is well-known to those skilled in the art. In addition, conditions for transfecting a plant with Agrobacterium harboring such a nucleic acid construct are also well-known to those skilled in the art.

In the present invention, other gene transfer methods may be also used. Examples of gene transfer methods include a method for introducing DNA into protoplast using polyethylene glycol or calcium, protoplast transformation procedures by electrophoration, a method using a particle gun, and the like.

The plant cells and the like manipulated as described above may be selected for transformation. This selection may be performed, for example, based on the expression of a marker gene present in the transformed nucleic acid construct. For example, when a drug resistance marker gene is used, selection can occur by cultivating or growing the plant cells in a culture medium which includes antibiotics or herbicide or the like at a moderate concentration. Alternatively, when a maker gene is a β-glucuronidase gene or a luciferase gene or the like, transformants can be selected by screening for the activity of the marker gene. If these identified transformants are other than the plant body such as protoplasts, calli, explants, or the like, a whole plant may be regenerated from them. For this regeneration, any method known to those skilled in the art can be used depending on the host plants to be used.

The plant thus obtained may be cultivated by a common method, that is, under the same conditions as for nontransformants, and in order to identify the transgenic plant containing a nucleic acid construct according to the present invention, various molecular biological methods can be used in addition to selection based on a marker gene described above. For example, Southern hybridization or PCR may be used to detect the presence or absence of a recombinant DNA insertion fragment and its structure. It is possible to use Northern hybridization or RT-PCR or the like to detect or measure a RNA transcript from an introduced nucleic acid construct DNA.

The expression of the introduced gene of the obtained transformant may then be evaluated by determining the amount of the protein, mRNA, or the enzyme activity, for example, of the GDH protein. For example, the amount of protein can be evaluated by the Western blot method and the like, and the amount of mRNA can be evaluated by the Northern blot method or the quantitative RT-PCR method. In addition, the above-described enzyme activity in the plant extracts may be measured by a usual method. For example, the GDH activity can be measured by the method of Ameziane et al. (Ameziane R., Bernhard K., and Lightfood D., Plant and Soil, 221: 47-57, 2000).

Accordingly, the 2-OG content in the transformed plant may be further evalutated by confirming expression of the introduced gene, for example, the GDH gene. The 2-OG content can be measured by, for example, an enzymatic method, whereby the whole or part of the transformed plant may be ground and an extracted solution prepared. The preparation of a plant extract and quantification of 2-OG may be performed by the method of Usuda (Usuda H., Plant Physiol, 78: 859-864, 1985). More specifically, samples may be prepared from a plant by an appropriate method. For example, 20 μl of extracted sample may be added to 475 μl of a reaction solution {0.1 M of Tris-HCl (pH 8.5), 1.0 mM of CaCl₂, 0.2 mM of NADPH, and 0.2 M of NH₄Cl}, the absorbance of the resulting solution may be measured at 340 nm, then 5 μl (10 units) of GDH may be added to the solution and the resulting solution may be allowed to react for 10 minutes at 37° C. After that, the absorbance may be measured again at 340 nm. The 2-OG content can be calculated by determining the difference between the absorbance measured after adding the enzyme solution and that measured before adding the enzyme solution. Under ordinary cultivation conditions, the 2-OG content is considered to be increased when the 2-OG content of above-ground part (leaf, stem, both of them, floral organ, or a part of fruit) or under-ground part (root or tuber) increases 1.2-fold or more when compared with that of the original line of the same species used for gene introduction (the nontransformed plant).

Two cases are considered for gibberellin quantification. One is the case where the gibberellin type for analysis is pre-determined. In this case, after necessary purification is performed, an instrumental analysis such as GC/MS method can be performed. The other is the case where the type(s) and the content of gibberellin(s) contained in the sample are not known. In this case, after purifying and/or fractionating the sample, a bioassay (biological assay) can be performed according to the method of, for example, Chen et al. For a bioassay, a drip method using dwarf mutant rice which responds sensitively to gibberellin, Tanginbozu, has been mainly used. In short, hulled unpolished rice seeds are surface sterilized by treating them with 70% ethanol for 1 minute and 2% sodium hypochlorite for 15 minutes, followed by washing with sterilized water, and then they are immersed in sterilized water and incubated for 2 days at 30° C. Then, the germinated seeds are again sterilized in the same manner, sowed on an agar medium (0.6% agar), and incubated at 25° C. for 5 days under daylight for 16 hours. Only the germinated seeds exhibiting uniform growth are selected, and 2 μl of the sample solution is dropped between the 1st leaf and the coleoptile. After cultivation for an additional 5 days under the same conditions, the gibberellin content may be determined by measuring the length of the 2nd leaf sheath. Gibberellin quantification can be performed by either instrumental analysis or bioassay. If the active gibberellin content is increased as compared with controls as determined by the instrumental analysis, or if the internode length is statistically significantly promoted (e.g., significant level of 5%) compared with controls as determined by the bioassay, it may be possible to determine that the gibberellin activity is increased.

Accordingly, once the transgenic plant, which has a decreased dormancy period due to an increase in endogenous 2-OG content and gibberellin activity compared with nontransformed plants as controls, is defined, the properties may be tested to determine whether they are genetically stable or not. To do this, the plants may be grown and cultivated under conventional conditions to obtain their seeds, or to obtain tubers, for example, in the case of a potato. The segregation of properties in their progeny may also be analyzed. The presence or absence of the introduced nucleic acid construct, its location, expression, and the like in the progeny may also be analyzed by a similar method as for the primary generation of transformants (T1 generation).

Although transgenic plants may be hemizygous or homozygous regarding the sequence of the nucleic acid construct introduced into the genome, it is possible to generate both hemizygotes and homozygotes in their progeny by crossing them, if required. The sequence of the nucleic acid construct integrated into the genome will segregate according to Mendelism in the progeny. Therefore, in order to obtain stable progeny plants and seeds, it is preferable to use homozygous plants.

In addition, in most transformants, a foreign gene is inserted in one locus, but it is not rare that the transformants are multi-copy transformants where the foreign gene has been inserted into a plurality of gene loci. For the sake of the introduced gene's stability etc., single-copy transformants are more preferable in the present invention.

Accordingly, the transgenic plants thus obtained are evaluated for dormancy period. The decrease in the dormancy period may be evaluated by comparing the plant dormancy period for each of the plant lines of the present invention with the dormancy period of the standard plant species, or by comparing the plant dormancy period for each of the plant lines produced by the present invention with the dormancy periods of the standard plant. The evaluation may be performed using a significant level of 5%. In general, this “standard plant species” is the “naturally occurring plant of the same species” as described previously.

For example, in the case of the transgenic potatoes, after harvesting, they are stored at room temperature (15 to 25° C.), or at a low temperature (4 to 8° C.) for about 3 months under conventional conditions, and then seed potatoes are planted in a culture soil. Then, the ratio of budding seed potatoes is compared with that of nontransgenic potatoes (for example, the source plant used for the introduction of a genetic construct according to the present invention, including the naturally occurring plant of the same species) to evaluate the decrease of the dormancy period. The actual days required for budding (sprouting) will vary depending on storage conditions, variety, cultivation conditions, and the like, but for example, if the transgenic plant and the nontransgenic plant originating from May Queen are stored at room temperature, 70% or more of the potatoes of the present invention or the potatoes produced by the present invention show a decrease in the period required for forcing germination by 1 to 8 weeks. Therefore, in the case of the potato, briefly, when the period required for forcing germination for 70% or more of potatoes under the conditions as described above is decreased by 1 to 8 weeks compared with the naturally occurring potatoes of the same species, it may also be considered that the dormancy period is decreased.

Accordingly, since the transgenic plants have early budding or germination timing and synchronized budding or germination compared with the nontransformant plant, it is possible to remarkably improve the growth or yield. Moreover, seeds can be obtained from the transgenic plant thus produced. The seeds can be easily produced by the same method as for the nontransgenic plant of the same species. If necessary, the seeds can be preserved, sterilized, disinsectizated and the like by conventional methods known to those skilled in the art.

In addition to the introduction of a specific nucleic acid construct, the dormancy period can be changed by increasing the plant intracellular 2-OG content through mutagenesis by radiation, ultraviolet ray, and/or mutagen (e.g., alkylating agents such as ethyleneimine or ethyl methane sulfonate) optionally followed by selection. In particular, independent of the introduction of a nucleic acid construct, the plant dormancy period can be shortened by increasing the intracellular 2-OG content by enhancing the expression of GDH gene, aspartate aminotransferase, and alanine aminotransferase. The general method for mutagenesis and reagents related to this method are well-known to those skilled in the art, which may be used for the present invention. The evaluation of the cultivation method, the 2-OG content, the amount of expression of each gene described above, and/or the reduced dormancy period of the plant thus produced can be performed according to the method and manner similar to those described for the transgenic plant.

As can be understood from the above description, according to the present invention, the dormancy period of the plant can be generally shortened as compared with the nontransformed plant, for example, the naturally occurring plants of the same species. The present invention may be applied to the plant for which gibberellin is effective in its dormancy breaking. Such plants include, but are not limited to, oats, lettuce, potato, peach, camellia, rice plant, barley, ramie, peanut, Bromus catharticus, Bromus inermis Leyss., radish, Brassica, lettuce, aubergine, beefsteak plant, gloxinia, kalanchoe, Primula, Nigella damascena, strawberry, and Aralia cordata Thunberg. A particularly preferable example of the plant having a decreased dormancy period according to the present invention and the plant produced by the present invention is a potato.

### EXAMPLES

The present invention will be described specifically and in detail by the following non-limiting examples, with regard to the production method of a plant having increased 2-OG content, particularly a plant manipulated for overexpression of GDH gene, measurement of 2-OG content, gibberellin assay, budding test, and yielding test.

### Example 1

### Isolation of NADP-Dependent GDH Gene from Aspergillus nidulans (A. nidulans) and Construction of Ti Plasmid Vector

Isolation of NADP-Dependent GDH Gene from Aspergillus nidulans (An-GDH) nidulans was inoculated on a potato dextrose agar medium, cultured at 30° C. overnight, and the resulting colonies were further cultured in a dextrose liquid medium for 2 days. The total RNA was obtained from these proliferated bacteria.

The mRNA was purified from the total RNA using Poly (A) Quick mRNA Isolation Kit (Stratagene Co.), and then first-strand cDNA was synthesized using First-strand cDNA Synthesis Kit (Amersham Bioscience Co.). A PCR reaction was conducted with the synthesized first-strand cDNA as a template using the PCR System 2400 manufactured by PerkinElmer as follows; 94° C.—3 min; 94° C.—45 sec, 59° C.—30 sec, 72° C.—90 sec, and 35 cycles; 72° C.—10 min. The primers were 5′-TCT AGA ATG TCT AAC CTT CCC GTT GAG-3′ (SEQ ID NO: 5) and 5′-GAG CTC TCA CCA CCA GTC ACC CTG GTC-3′ (SEQ ID NO: 6). As a result, a band was found of about 1.4 kbp, which is consistent with the expected size of the intended gene. Obtained PCR products were cloned using TA-Cloning-Kit (Invitrogen Co.). Nucleotide sequence of the obtained clone was determined using a sequencer (377A of ABI Co.). The nucleotide sequence is shown in SEQ ID NO: 1 and the amino acid sequence of the encoded protein is shown in SEQ ID NO: 2.

This sequence coincided with the known nucleotide sequence of NADP-dependent GDH gene from A. nidulans, which was designated as An-GDH.

(2) Construction of Ti Plasmid Vector

Then, a nucleotide sequence encoding the transit peptide for mitochondria was connected to the obtained genes. A nucleotide fragment encoding the transit peptide for mitochondria was obtained by conducting PCR using the 5′ side of NAD-dependent GDH gene from tomatoes as a template. The primers for the PCR reaction were 5′-CTG CAG ATG AAT GCT TTA GCA GCA AC-3′(SEQ ID NO: 7) and 5′-TCT AGA TAA ACC AAG AAG CCT AGC TG-3′(SEQ ID NO: 8). The connection of the An-GDH gene and the nucleotide sequence encoding the transit peptide for mitochondria was conducted by PCR with the An-GDH gene and transit peptide gene as templates. The four primers for the PCR reaction were 5′-TCT AGA ATG AAT GCT TTA GCA GCA AC-340 (SEQ ID NO: 9), 5′-GGG AAG GTT TAG ACA TTA AAC CAA GAA GCC T-3′(SEQ ID NO: 10), 5′-AGG CTT CTT GGT TTA ATG TCT AAC CTT CCC-3′(SEQ ID NO: 11) and 5′-GAG CTC TTA CGC CTC CCA TCC TCG AA-3′(SEQ ID NO: 12). The transit peptide sequence for mitochondria was added to the obtained GDH gene, and designated Mtd-An-GDH. The Mtd-An-GDH gene was incorporated into pIG121-Hm by replacing the GUS part of pIG121-Hm, a vector, and the Ti-plasmid for Agrobacterium mediated transformation (FIG. 1). The obtained Ti-plasmid was introduced into Agrobacterium tumefaciens EHA101, and used for potato transformation.

### Example 2

### Production of Transgenic Potato

The transformation of a potato (May Queen) was performed by the method of Gordon et al. (Plant Cell Reports, 12: 324-327, 1993). Microtubers were induced in a sterile environment, cut to form tuber disks, and the pieces were placed on an MS agar medium including 2 mg/l of Zeatin and 0.1 mg/l of indoleacetic acid. The pieces were cultured at 25° C. for 24 hours with daylight hours kept to 16 hours. YEP medium (10 g/l of bactotrypton, 10 g/l of Yeast Extract and 1 g/l of glucose) containing 50 mg/l of kanamycin and 50 mg/l of Hygromycin was inoculated with Agrobacetrium containing constructed nucleic acid molecules and cultured overnight at 28° C. with shaking. The Agrobacterium suspension was added to the tuber disks which had been cultured for 24 hours to cause the infection. In 10 minutes, superfluous Agrobacterium suspension was removed using sterilized filter paper, transferred into a Petri dish containing the above-described medium, and cultured for 24 hours under the same conditions. Then, to select the transformants, the tuber disks were transplanted in the MS agar medium containing 50 mg/l of kanamycin, 300 mg/l of Cefotaxime hydrochloride, 2 mg/l of Zeatin, and 0.1 mg/l of indoleacetic acid. The regenerated shoot was further transferred into the above-described medium and its resistance was confirmed. The shoot exhibiting apparent kanamycin resistance was transplanted in an MS agar rooting medium containing 50 mg/l of kanamycin and 300 mg/l of Cefotaxime hydrochloride to induce root differentiation. The cane top part was cut at least 3 times from the redifferentiated rooting plant, and transplanted on a regeneration-selection medium to confirm kanamycin resistance, thereby excluding chimeras. A total of 5 individuals thus obtained were acclimated to the soil to obtain tubers.

### Example 3

### Confirmation of Introduced Gene

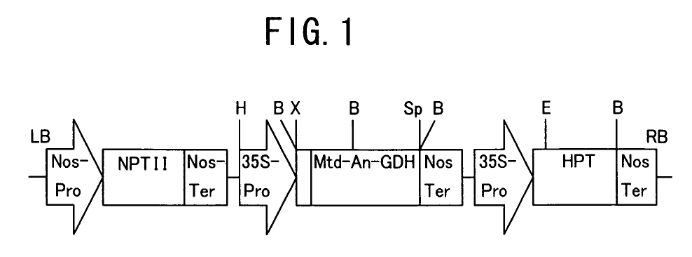

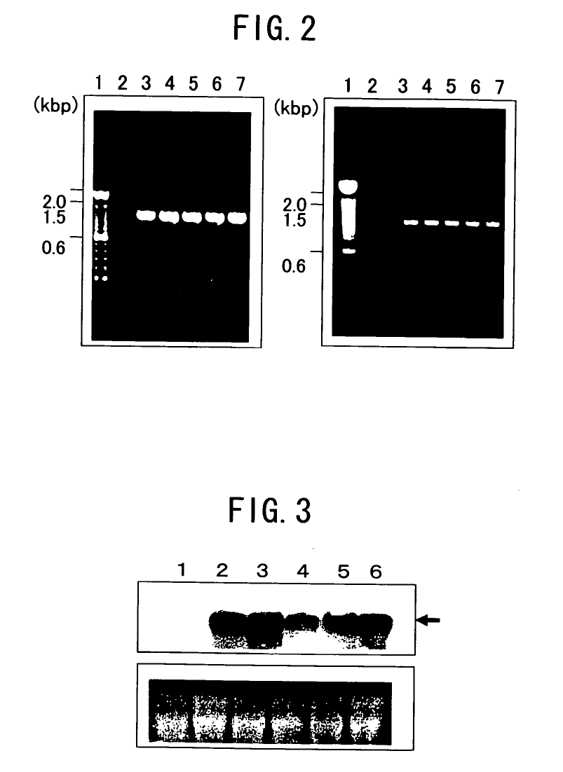

DNA was extracted from 5 selected individual lines having kanamycin resistance and non-infected plants. DNA was extracted by the method of Honda et al. (Honda and Hirai, Jpn. J Breed 40: 339-348, 1990). PCR analysis was conducted with the extracted DNA as a template, for An-GDH gene specific primers, 5′-TCT AGA ATG TCT AAC CTT CCC GTT GAG-3′(SEQ ID NO: 5) and 5′-GAG CTC TCA CCA CCA GTC ACC CTG GTC-3′(SEQ ID NO: 6), and primers for amplifying NPTII gene in a vector, 5′-CCC CTC GGT ATC CAA TTA GAG-3′(SEQ ID NO: 13) and 5′-CGG GGG GTG GGC GAA GAA CTC CAG-3′(SEQ ID NO: 14). The PCR reaction was conducted using the PCR System 2400 manufactured by PerkinElmer Co. as follows: 94° C.—3 min; 94° C.—45 sec, 55° C.—30 sec, 72° C.—90 sec, 35 cycles; 72° C.—10 min. The PCR product was subjected to electrophoresis on 1% agarose gel and then stained with ethidium bromide to examine the presence or absence of an amplification product and the size thereof. As a result, specific bands for the An-GDH gene (about 1.5 kbp) and the NPTII gene (about 1.1 kbp) were observed in the selected transgenic potatoes (FIG. 2), and these bands were not observed in non-infected plants. As a result, it was confirmed that the T-DNA region including An-GDH gene was introduced into the transformed potatoes.

### Example 4

### Confirmation of Expression of the Introduced Gene by Northern Blot Analysis

The expression of the introduced gene was confirmed by conducting a Northern blot analysis using transgenic potatoes in which the introduction of the An-GDH gene had been confirmed.

RNA was extracted from the tubers of transformed potatoes by a SDS-phenol method. The extracted RNA was subjected to electrophoresis on 1.2% agarose gel containing 18% formaldehyde and then stained with ethidium bromide. After it was blotted on a nylon membrane (HybondN+), it was fixed with UV, and hybridization was conducted with a probe containing the full length of An-GDH gene. DIG-High Prime DNA Labeling and Detection Starter Kit II and PCR DIG Probe Synthesis Kit manufactured by Roche Diagnostics K.K. were used for Northern blot and probe preparation.

As a result, an An-GDH gene specific band was confirmed for the tubers of the transgenic potatoes (FIG. 3). The expression of the introduced gene was shown in leaf tissues and tubers of the transgenic potatoes. This An-GDH specific band was not observed in nontransformed potatoes.

### Example 5

### Expression of the Introduced Gene (NADP-GDH Activity)

The NADP-GDH activity was determined to confirm the expression of the introduced gene using transformed potatoes in which the introduction of An-GDH gene had been confirmed.

The measurement for activity was conducted by the method of Ameziane et al. (Plant and Soil, 221: 47-57, 2000). Leaf tissues (about 0.2 g) of the transgenic potatoes were frozen with liquid nitrogen, and then crushed in a mortar. An extract buffer {200 mM of Tris (pH 8.0), 14 mM of β-mercaptoethanol, 10 mM of L-cysteine-HCl, and 0.5 mM of PMSF} of 5 volumes of the tissue sample was added thereto. The obtained suspension was transferred into a centrifugal tube and centrifuged at 12,000 rpm at 4° C. for 30 minutes. The supernatant was ultrafiltrated (Millipore, ultrafree 0.5 filter unit, Biomax-10) and washed with the extract buffer three times to recover the sediment on the filter and prepare a crude enzyme solution.

The activity was measured by adding the previously extracted crude enzyme solution to a reaction solution containing 100 mM of Tris (pH 8.5), 20 mM of 2-oxoglutarate, 10 mM of CaCl₂, 0.2 mM of NADH, and 200 mM of NH₄Cl, and the absorbance was measured at 340 nm. The protein concentration of the extracted crude enzyme solution was measured by the Bradford method using bovine serum albumin (BSA) as the standard.

As a result, 20- to 50-fold higher activity was observed in the sample from transgenic potatoes compared with that from nontransgenic potatoes (Table 1). This activity was considered to be due to the expression of the introduced An-GDH gene, since there was little NADP-GDH activity in the sample from nontransformed potatoes.

TABLE 1
NADP-GDH activity in transgenic potato and nontransgenic potato

| | NADP-GDH activity (μmol/min/mg protein) |
|---|---|
| Nontransgenic | 0.11 |
| Transgenic | |
| Mtd 2 | 2.12 |
| Mtd 5 | 4.64 |
| Mtd 8 | 5.85 |

### Example 6

### Determination of 2-oxoglutarate (2-OG) Contents of GDH Gene-Introduced Potatoes

The 2-OG content in the leaf tissues of transgenic potatoes (Mtd 8) was determined. Potatoes were cultivated for 1 month in a mixed soil of 500 g Power Soil and 500 g vermiculite to obtain healthy, grown plants, and its leaf tissue was used as a test material. Extraction was performed by the method of Agarei et al. (Plant Science, 162: 257-265, 2002). After the leaf tissue (about 0.1 g) was crushed with liquid nitrogen, 200 μl of 3% HCO₄ was added thereto and mixed well. After the centrifugation at 12,000 rpm for 10 minutes, the supernatant was transferred into another tube. To the remaining sediment, 200 μl of 3% HCO₄ was added and mixed well. After centrifugation at 12,000 rpm for 10 minutes, the supernatant was transferred into the former tube. Then, 50 μl to 70 μl of 5 M of K₂CO₃ was added to the recovered supernatant for neutralizing the solution. After confirming that the pH of the solution was neutral using litmus paper, sterilized water was added to the solution to adjust the volume to 600 μl and the resultant solution was used as a test sample. The determination of 2-OG content was conducted according to the modified method of Usuda et al (reference: previously quoted). The extracted sample of 20 μl was added to 475 μl of a reaction solution {0.1 M of Tris-HCl (pH 8.5), 1.0 mM of CaCl₂, 0.2 mM of NADPH, and 0.2 M of NH₄Cl}. After the absorbance was measured at 340 nm, 5 μl (10 units) of glutamate dehydrogenase (GDH) was added thereto and the resulting solution was reacted at 37° C. for 10 minutes. The absorbance at 340 nm was measured again and the 2-OG content was calculated by using the difference between the absorbance value measured after the enzyme solution was added and that measured before the enzyme solution was added.

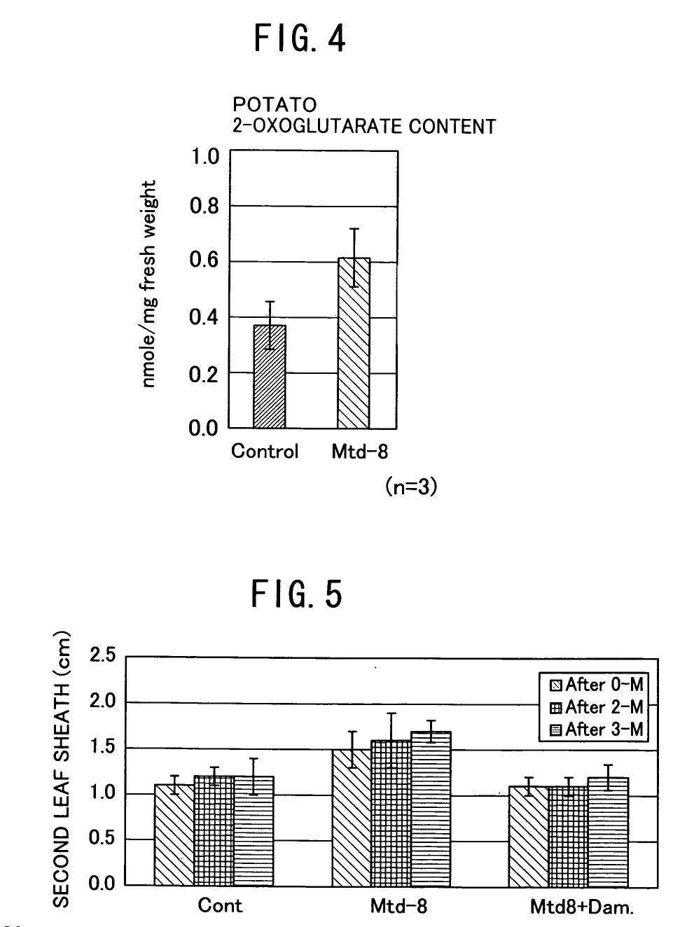

As a result, in the transformed potatoes, the 2-OG content was increased 1.7-fold compared with that of nontransformed potatoes (FIG. 4).

### Example 7

### Gibberellin Bioassay for GDH Gene-Introduced Potato Tubers

A dwarf mutant of rice, tanginbozu. was used in an assay. The assay method was based on the method of Chen et al. (Plant Cell and Environment, 24: 469-476, 2001). Dehusked unpolished rice seeds were surface-sterilized with 70% ethanol for 1 minute and 2% sodium hypochlorite for 15 minutes followed by washing with sterilized water 3 times, immersed in sterilized water, and incubated at 30° C. for 2 days. Germinating seeds were sterilized again in a manner similar to that described above. After 0.6% agar plates were added to distilled water and sterilized by autoclave, germinating seeds were placed in test tubes of 3 cm in diameter, and each filled with agar medium of 25 ml. This was cultured at 25° C. for 5 days with daylight hours kept to 16 hours. Only the germinating seeds having uniform seedlings were selected, and 2 μl of test solution was dropped between the 1st leaf and the coleoptile. After being cultured under the same condition for 5 days, the length of the 2nd leaf sheath was measured.

For the assay, tubers which were stored at −80° C. immediately after harvesting, tubers which were 2 months post-harvest, and tubers which were 3 months post-harvest were used. Harvested tubers were stored indoors. All the tubers were crushed using liquid nitrogen, and extracted by adding 80% acetone at 3×-volume of the fresh weight. After impurities were removed by centrifugation, the supernatant was freeze-dried. 20% acetone was added to the freezed-dried sample to give the fresh weight of 100 μl/g. To examine the specificity of gibberellin, 1 mg/l of daminozide, a gibberellin antagonist, was used. The following 3 groups were prepared: a test solution from transgenic potato tubers, a test solution from nontransgenic potato tubers, and a test solution of transgenic potato tubers which had the same amount of daminozide added thereto.

As a result, it was confirmed that in the test solution extracted from the transgenic potato tubers, the extension length of the 2nd leaf sheath was enhanced as compared with that extracted from the nontransgenic potato tubers. This suggested that the content of active gibberellin was increased in the test solution from the transgenic potato tubers as compared with that from the nontransgenic potato tubers. Moreover, since the effect of the extension promotion on the 2nd leaf sheath was inhibited by the addition of daminozide, the extension of the 2nd leaf sheath was considered a result of the gibberellin-specific effects (FIG. 5). Furthermore, since the extension promotion on the 2nd leaf sheath length appeared to be enhanced as the period of tuber storage increased, it was considered that the dormancy would be broken by a gradual increase in the gibberellin activity within the tubers during long-term storage.

### Example 8

### Study on Budding of Transformed Potatoes

Budding from transgenic potato tubers and nontransgenic potato tubers of 3 months after harvesting was studied. May budding tubers were observed for transgenic potatoes (40 out of 42 tubers), but no budding tubers were observed for nontransgenic potatoes (0 out of 30 tubers). Moreover, after the light treatment, budding treatment was performed for 10 transgenic potato tubers and 10 nontransgenic potato tubers at room temperature, 25° C., for 3 days. One group was transplanted in a soil composed only of vermiculite, and the other group which had the same number of tubers was kept as they were. Then, the number of buds was determined. The transplanted tubers were cultured in a closed system greenhouse maintained at 24° C. during the day and at 18° C. during the night, and the number of tubers having buds sprouting above the ground was measured each week.

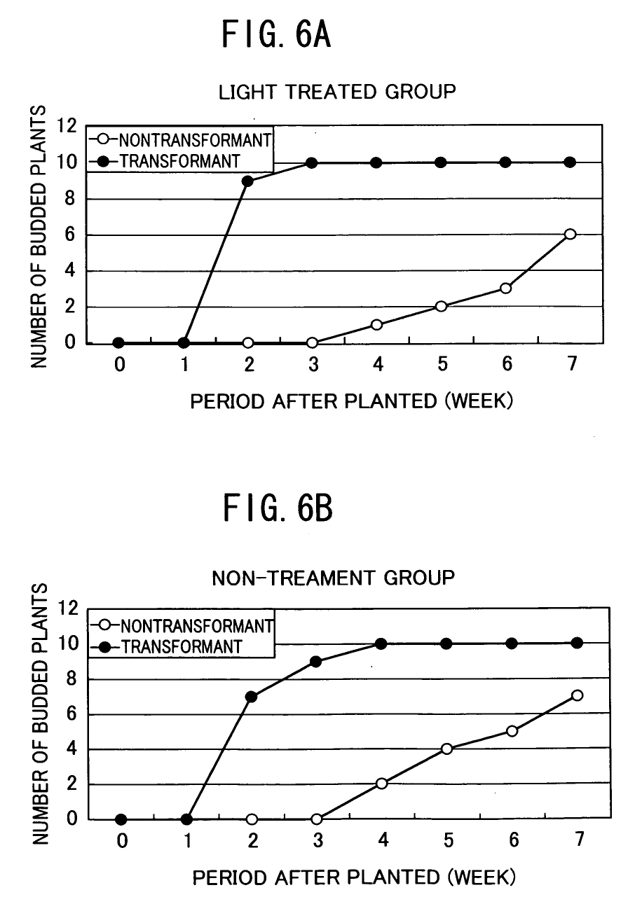

As a result, budding for transgenic potato tubers was observed 2 weeks after transplantation, and all the tubers budded 3 weeks after transplantation for the transgenic potato tubers. On the contrary, in nontransgenic potatoes, some budding was observed in the 4th week, but budding from all the tubers was not observed even after the 7th week (FIG. 6). From these results, it was confirmed that the budding timing of transgenic potato tubers was advanced and that the budding occurred very uniformly.

### Example 9

### Study on the Yield of Transformed Potatoes

The yield was studied using the tubers of transgenic potatoes and nontransgenic potatoes. 0.3 kg Power Soil (Kureha Chemical Industry Co.) and 1 kg vermiculite were added to a No. 7 pot and one tuber was placed in each pot. In the study, 8 tubers of transgenic potatoes and 8 tubers of nontransgenic potatoes were cultivated. The fresh weight of the above-ground part, and the number of tubers and tuber weight were measured. During cultivation, the number of stems was normalized to one per tuber and only water was given without additional fertilizer.

As a result, the weight of the above-ground part, the tuber weight, and the number of tubers of transgenic potatoes increased 1.13-fold, 1.35-fold, and 1.24-fold, respectively, compared with those of nontransgenic potatoes (Table 2).

TABLE 2
Study on the yield of transformed potatoes containing introduced
GDH gene

| | Nontransgenic potato | Transgenic potato |
|---|---|---|
| Fresh weight of | 11.9 ± 2.1 (1.00) | 13.5 ± 2.4 (1.13) |
| above-ground part (g) | | |
| Tuber weight (g) | 41.0 ± 7.0 (1.00) | 55.4 ± 6.5 (1.35) |
| Number of tubers | 2.5 ± 0.5 (1.00) | 3.1 ± 1.1 (1.24) |

References

1. Canada, CA2180786

2. China, CN00109779.2

3. Japanese Unexamined Patent Publication No. 2001-238556

4. Tomokazu Koshiba and Yuji Kamiya, “Science of new plant hormone”, Kodansha Ltd., 2002

5. Vegetable horticulture great dictionary, Yokendo, 1977

6. Tokushima Test and Research Report of Agriculture, vol. 36, pp. 7-17 (2000)

7. Minoru Yoshida, Potato encyclopedia, Rural Culture Association, 1988

8. Plant Physiology, vol. 118, pp. 773-781 (1998)

9. Plant Journal, vol. 22, pp. 247-256 (2000)

While the invention has been described in detail with reference to preferred embodiments thereof, it will be apparent to one skilled in the art that various changes can be made, and equivalents employed, without departing from the scope of the invention. Each of the aforementioned documents, including Japanese Patent Appln. No. 2004-039659 filed on Feb. 17, 2004, is incorporated by reference herein in its entirety.

1. A method for producing a plant comprising increasing the intracellular 2-OG content in said plant, and selecting a plant which has a decreased dormancy period as compared with the dormancy period of a naturally occurring plant of the same species.

2. The method according to claim 1, wherein said plant has uniform budding and/or germination timing.

3. A method for producing a plant comprising overexpressing an intracellular GDH gene in said plant, and selecting a plant which has a decreased dormancy period as compared with the dormancy period of a naturally occurring plant of the same species.

4. The method according to claim 3, wherein said plant has uniform budding and/or germination timing.

5. The method according to claim 3, wherein a nucleic acid construct capable of enhancing the expression of the GDH gene is introduced into said plant.

6. The method according to claim 3, wherein a nucleic acid construct capable of expressing the GDH gene is introduced into said plant.

7. The method according to claim 5, wherein said nucleic acid construct comprises a nucleic acid molecule encoding the polypeptide comprising the amino acid sequence selected from the group consisting of SEQ ID NO: 2 and SEQ ID NO: 4.

8. The method according to claim 5, wherein said nucleic acid construct hybridizes under stringent conditions to a nucleic acid molecule selected from the group consisting of SEQ ID NO: 1 and SEQ ID NO: 3, and wherein said nucleic acid molecule encodes a polypeptide having GDH activity.

9. The method according to claim 1, wherein said plant is selected from the group consisting of oat, lettuce, potato, peach, camellia, rice, wheat, ramie, peanut, Bromus catharticus, Bromus inermis Leyss, Japanese radish, Brassica, aubergine, beefsteak plant, gloxinia, kalanchoe, Primula, Nigella damascena, strawberry, Aralia cordata Thunberg, onion, scallion, spinach, and flower bulb.

10. The method according to claim 9, wherein said plant is a potato.

11. A plant which has an increased intracellular 2-OG content and a decreased dormancy period as compared with the intracellular 2-OG content and dormancy period of a naturally occurring plant of the same species.

12. The plant according to claim 11, wherein said plant has uniform budding and/or germination timing.

13. A plant which has a decreased dormancy period as compared with the dormancy period of a naturally occurring plant of the same species, wherein said plant overexpresses an intracellular GDH gene.

14. The plant according to claim 13, wherein said plant has uniform budding and/or germination timing.

15. The plant according to claim 13, wherein said GDH gene comprises a nucleotide sequence encoding the polypeptide comprising the amino acid sequence selected from the group consisting of SEQ ID NO: 2 and SEQ ID NO: 4.

16. The plant according to claim 13, wherein said GDH gene comprises a sequence capable of hybridizing under stringent conditions to the nucleic acid molecule selected from the group consisting of SEQ ID NO: 1 or SEQ ID NO: 3, and wherein said gene encodes a polypeptide having GDH activity.

17. The plant according to claim 11, wherein said plant is selected from the group consisting of oat, lettuce, potato, peach, camellia, rice, wheat, ramie, peanut, Bromus catharticus, Bromus inermis Leyss., Japanese radish, Brassica, aubergine, beefsteak plant, gloxinia, kalanchoe, Primula, Nigella damascena, strawberry, Aralia cordata Thunberg, onion, scallion, spinach, and flower bulb.

18. The plant according to claim 17, wherein said plant is a potato.


SEQUENCE LISTING (14 sequences)

SEQ ID NO: 1 - DNA, 1377 bp, Aspergillus nidulans
  CDS (1)..(1377)
atgtctaaccttcccgttgagcccgagttcgagcaggcctacaaggagcttgcgtcgaccctcgagaactccaccctctttgagcagcaccctgaataccgacgggctctccaggtcgtctccgttcccgagcgcgttatccagttccgtgtcgtttgggagaacgacaagggcgaggttcagatcaaccgcggttaccgtgttcagttcaactccgctctcggtccctacaagggtggtctccgtttccacccctccgtcaacctttctatcctgaagttccttggcttcgagcagatcttcaaaaatgctctcacaggactaaacatgggtggtggcaagggtggttccgacttcgaccccaagggcaagtctgactctgaaattcgtcgcttctgtaccgctttcatgactgagctctgcaagcacatcggcgcggacactgaccttcccgctggtgatatcggtgttactggccgtgaggttggtttccttttcggccagtaccgcaggatccgcaaccagtgggagggtgttctcactggcaagggtggcagctggggtggtagcttgatccgccctgaagccactggatacggtgttgtctactacgttcagcacatgatcaagcacgttaccggtggaaaggagtccttcgcaggcaagcgtgtcgccatctccggctccggtaacgttgcccagtacgccgctctcaaggtcatcgagctcggtggttccgttgtctccctttccgactccaagggctctctcattgtcaaggatgagtccgcttctttcacccctgaagagatcgccctcattgccgacctcaaggttgcccgcaagcaactctccgagctcgccacctcctccgctttcgccggcaagttcacctacatccccgatgctcgcccttggaccaacattcccggcaagttcgaggttgctctcccttctgccactcagaacgaagtctccggcgaggaagccgagcacctcatcaagtccggtgtccgctatattgctgagggttccaacatgggttgcacccaggccgccatcgacatctttgaggctcaccgcaacgccaaccccggcgatgccatctggtacgcccctggtaaagccgccaacgctggtggtgtcgccgtctctggtcttgagatggctcagaactctgctcgtctctcctggacatccgaggaggtcgatgctcgcctcaagggcatcatggaggactgcttcaagaacggtctcgagactgctcagaagttcgctactcctgccaagggcgtcctgccttccctcgtcaccggttccaacattgccggtttcaccaaggtcgccgaggccatgaaggaccagggtgactggtgg

SEQ ID NO: 2 - protein, 459 aa, Aspergillus nidulans
MSNLPVEPEFEQAYKELASTLENSTLFEQHPEYRRALQVVSVPERVIQFRVVWENDKGEVQINRGYRVQFNSALGPYKGGLRFHPSVNLSILKFLGFEQIFKNALTGLNMGGGKGGSDFDPKGKSDSEIRRFCTAFMTELCKHIGADTDLPAGDIGVTGREVGFLFGQYRRIRNQWEGVLTGKGGSWGGSLIRPEATGYGVVYYVQHMIKHVTGGKESFAGKRVAISGSGNVAQYAALKVIELGGSVVSLSDSKGSLIVKDESASFTPEEIALIADLKVARKQLSELATSSAFAGKFTYIPDARPWTNIPGKFEVALPSATQNEVSGEEAEHLIKSGVRYIAEGSNMGCTQAAIDIFEAHRNANPGDAIWYAPGKAANAGGVAVSGLEMAQNSARLSWTSEEVDARLKGIMEDCFKNGLETAQKFATPAKGVLPSLVTGSNIAGFTKVAEAMKDQGDWW

SEQ ID NO: 3 - DNA, 1113 bp, Aspergillus awamori
  CDS (1)..(1113)
atgtctaaccttcctcacgagcccgagttcgagcaggcctacaaggagcttgcctcgacccttgagaactccaccctcttccagaagaaccccgaataccgcaaggcccttgctgtcgtctccgtccccgagcgtgtcatccagttccgtgtcgtctgggaggatgatgccggcaacgtccaggtcaaccgcggtttccgtgtccagttcaacagcgccctcggtccctacaagggtggtcttcgtttccacccctccgtcaacttgtccatcctccaagttccttggtttcgagcagatcttcaagaatgctctcactggcctgaacatgggtggtggtacgtcgagcacatgattgctcacgccaccaacggccaggagtccttcaagggcaagcgcgttgccatctccggttccggtaacgttgcccagtacgccgccctcaaggtcattgagctcggcggttccgtcgtctccctgagcgacacgcagggctccctcatcatcaacggcgagggtagcttcacccccgaggagatcgagctcatcgctcagaccaaggtcgagcgcaacgagctcgccagcatcgtcggtgctgctcccttcagcgacgccaacaagttcaagtacattgctggtgcccgcccctgggttcacgtcggcaaggtcgacgtcgctctcccctccgctacccagaacgaagtttccggcgaggaggcccaggtcctcatcaacgctggctgcaagttcatcgccgagggttccaacatgggttgcacccaggaggccatcgacaccttcgaggcccaccgtaccgccaacgctggcgcggctgccatctggtacgcccccggtaaggccgccaacgccggtggtgtcgctgtctccggtctggagatggctcagaactctgcccgcctcagctggacttctgaggaggttgatgcccgtcttaaggacatcatgcgcgactgcttcaagaacggtcttgagactgctcaggagtacgccacccccgctgagggtgtcctgccttccctggtgaccggatccaacattgccggtttcaccaaggtggctgccgccatgaaggaccagggtgactggtggtaa

SEQ ID NO: 4 - protein, 370 aa, Aspergillus awamori
MSNLPHEPEFEQAYKELASTLENSTLFQKNPEYRKALAVVSVPERVIQFRVVWEDDAGNVQVNRGFRVQFNSALGPYKGGLRFHPSVNLSILQVPWFRADLQECSHWPEHGWWYVEHMIAHATNGQESFKGKRVAISGSGNVAQYAALKVIELGGSVVSLSDTQGSLIINGEGSFTPEEIELIAQTKVERNELASIVGAAPFSDANKFKYIAGARPWVHVGKVDVALPSATQNEVSGEEAQVLINAGCKFIAEGSNMGCTQEAIDTFEAHRTANAGAAAIWYAPGKAANAGGVAVSGLEMAQNSARLSWTSEEVDARLKDIMRDCFKNGLETAQEYATPAEGVLPSLVTGSNIAGFTKVAAAMKDQGDWW

SEQ ID NO: 5 - DNA, 27 bp, Artificial
  PCR Primer
tctagaatgtctaaccttcccgttgag

SEQ ID NO: 6 - DNA, 27 bp, Artificial
  PCR Primer
gagctctcaccaccagtcaccctggtc

SEQ ID NO: 7 - DNA, 26 bp, Artificial
  PCR Primer
ctgcagatgaatgctttagcagcaac

SEQ ID NO: 8 - DNA, 26 bp, Artificial
  PCR Primer
tctagataaaccaagaagcctagctg

SEQ ID NO: 9 - DNA, 26 bp, Artificial
  PCR Primer
tctagaatgaatgctttagcagcaac

SEQ ID NO: 10 - DNA, 31 bp, Artificial
  PCR Primer
gggaaggtttagacattaaaccaagaagcct

SEQ ID NO: 11 - DNA, 30 bp, Artificial
  PCR Primer
aggcttcttggtttaatgtctaaccttccc

SEQ ID NO: 12 - DNA, 26 bp, Artificial
  PCR Primer
gagctcttacgcctcccatcctcgaa

SEQ ID NO: 13 - DNA, 21 bp, Artificial
  PCR Primer
cccctcggtatccaattagag

SEQ ID NO: 14 - DNA, 24 bp, Artificial
  PCR Primer
cggggggtgggcgaagaactccag